(19) United States
(12) Patent Application Publication
(10) Pub. No.: US 2005/0089534 A1
(43) Pub. Date: Apr. 28, 2005

(54) Lipid enriched domains as vaccine targets in tumor therapy

(76) Inventors: Jan Holmgren, Vastra Frolunda (SE); Pam Fredman, Goteborg (SE)

Correspondence Address: Matthew E. Connors;Gauthier & Connors LLP, Suite 3300, 225 Franklin Street, Boston, MA, 02110, US

(21) Appl. No.: 10/639,667

(22) Filed: Aug. 12, 2003

Related U.S. Application Data
(60) Provisional application No. 60/403,421, filed on Aug. 14, 2002.

(51) Int. Cl. A61K 39/00

(52) U.S. Cl. 424/277.1

(57) ABSTRACT

The present invention relates to immunogenic rafts, lipid enriched membrane domains, and their use in cancer therapy.

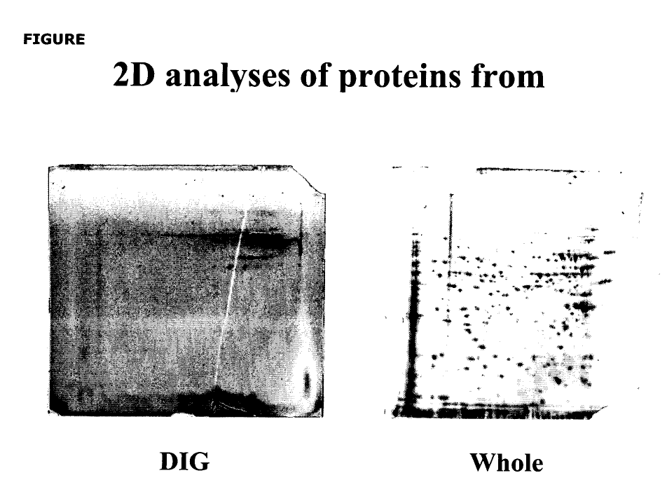

### TECHNICAL FIELD

The present invention relates to method for treating cancer using so called rafts, the use of such rafts in the manufacture of therapeutically immunogenic compositions, as well as a method for isolating such rafts.

### BACKGROUND OF THE INVENTION

Cancer

Cancer is a leading cause of death in the world, and the incidence is still increasing owing to changes in life style and longer life expectancy. It is the second leading cause of death in the United States with an estimated 563,100 cancer deaths in 1999 and 1,222,000 new cancer cases diagnosed each year. Improvements in the treatment of cancer have been achieved in the last decades but the prognoses for many cancers still remains poor. Thus there is a need of developing new treatment strategies.

Immunotherapy of Cancer

Among various strategies to develop more efficient tumor treatment is to utilize immunotherapy. There is substantial evidence indicating that the immune system plays a critical role in the prevention of cancer and the control of tumor growth. This includes the occasional observation of spontaneous tumor regression, the correlation of spontaneous regressions with the presence of tumor-infiltrating lymphocytes (TILs) and the identification of TILs that are specific for tumor antigens. However, as evidenced by the incidence rates of cancer, the immune response is often not sufficient to successfully combat the tumor. During the past 40 years, an increased understanding of the immune mechanism has led to the development of approaches to enhance the immune response against the tumor. These have included the expression in tumor cells of genes encoding immunostimulatory molecules and several approaches to enhance tumor antigen presentation. Unfortunately, only limited indications of beneficial effects have been seen and attempts to utilize these new approaches to immunotherapy have been disappointing.

Tumor Antigens in “Lipid Enriched Membrane Domains”

A substantial number of potential tumor antigens have been described and utilized experimentally for immunotherapy development purposes and include mainly various proteins/peptides but also specific glycosphingolipids. There is substantial evidence that the same T cell-defined tumor antigens are expressed by different human tumors, suggesting that transformation-associated events may give rise to recurrent expression of the same tumor antigen in tumors of related tissue and/or cellular origin (Shamamian et al., Cancer Immunol. Immunother., 1994;39:73-83; Cox et al., Science, 1994;264:716). Examples of such tumors include solid and disseminated tumors, but are not limited to lung, colorectal, pancreatic, prostate, ovarian, breast, multiple myeloma, leukemias and melanoma. Examples of tumor-associated antigens include but are not limited to HER2/neu or c-erbB-2, HER3 and HER4 (breast), MAGE-1 and MAGE-3 (bladder, head and neck, non-small cell carcinoma); MART 1/Melan A, gp-100, and tyrosinase (melanoma); carcino-embryonic antigen (CEA-colon, breast, gastrointestinal tract); MUC-1 complex antigens including 19-9 and CA242 (breast, pancreas, colon and prostate); prostate-specific membrane antigen (PSMA); HPV E6 and E7 proteins (cervical cancer); tumor-associated heat-shock protein antigens, and various glycolipid antigens with sugar specificity including fucosyl-GM1, GD3, and sialyl T (Hakomori, Cancer Res. 1985; 45:2405-14; Nilsson et al., Glycoconjugate J. 1984; 1: 43-49; Nilsson et al., Cancer Res. 1986; 46: 140301407; Fredman et al., Biochim. Biophys. Acta 1986; 875: 316-323).

The tumor antigens described supra can be obtained synthetically or from whole irradiated tumor tissue or cells, from tumor tissue or cell extracts, or from tumor tissue or cell membrane preparations as described (Herlyn and Koprowski, Ann. Rev. Immunol. 1988; 6: 283-308; Schreiber et al., Ann Rev. Immunol. 1988; 6: 359-80; Screiber, Fundamental Immunol. Ed. W. E. Paul 1999, pp. 1237-1270, Lippincott-Raven, Philidelphia). These antigens, irrespective of their chemical nature, have however to date always been studied as single molecules and without consideration of possible influence of them as immunogens acting in combination with each other. During the last decade there is an increasing body of evidence for the importance of lipid enriched membrane domains, so called “rafts”, as key cellular sites for expressing important cell recognition structures on the cell surface, for interacting with the surrounding extracellular milieu as well as with adjacent cells, and for initiating important cell signaling functions. Molecular biology technology including the development of transgenic mouse models have recently made considerable contributions to defining the many important roles of “rafts” and to the awareness that associated membrane lipids play a crucial role in maintenance of normal cell function (1-3). “Rafts” form functional protein-lipid domains (4,5) and besides a general increase of cholesterol in rafts glycosphingolipids are particularly enriched in these membrane domains.

Rafts are formed by lipid-lipid intramembrane interaction and the strength of such interaction depending on the physicochemical properties of the lipid moieties and such properties are found in glycosphingolipids which cluster in rafts. These rafts might be isolated by homogenization of cells/tissues in buffers that challenge the in vivo lipid-lipid interaction. The names of these isolated rafts are varying but are in this application referred to as DIGs (detergent insoluble membrane domains) to reflect the main methodology for their isolation.

Glycosphingolipids are membrane lipid constituents expressed in all mammalian cells and structural variability as well in the carbohydrate as in the lipid composition imply biological function and physico-chemical property diversity. It is well established (refs) that tumor cells not only express tumor specific proteins but also structurally altered glycosphingolipids. Glycosphingolipids might not only be of immunological interest as antigens. They have also, in several studies, been shown to induce cytokine response either as receptors (24) or by direct effect.

### THE INVENTION

The goal of immunotherapy is to augment a patient's immune response to an established tumor. The present invention is based on the demonstration that rafts can contain tumor antigens of interest for immunotherapy and that rafts may therefore be a preferred source of isolation of antigens for immunotherapy and in an especially preferred embodiment even be used directly as immunogen, alone or together with immunostimulatory agents or formulations, to trigger an anti-tumor immune response. The present invention includes both the use of rafts as immunogen or as a source of antigen for use in cancer immunotherapy and methods to identify and isolate membrane domains, rafts, containing tumour proteins and/or glycosphingolipids, and to utilize such rafts or their contained antigens as immunogens for cancer immunotherapy.

Rafts/DIGs—Identity

In this application rafts/DIGs refer to lipid enriched membrane domains, ie cell membrane domains with increased density of lipids, formed by lipid-lipid interactions and having physical-chemical properties that differ from surrounding membrane areas.

Methodology

Isolation of DIGs

Cells or tissue specimens or cellular membranes therefrom (fresh or frozen and thawed) are homogenized in solutions (detergent/buffer) preserving DIGs as identified above. DIGs are then separated from non-DIGs fractions by any methodology, for example sucrose gradient, where the lipid enriched characteristics are utilized. Isolated DIGs might be stored frozen until analysis are being performed.

Preparation of Protein and Lipid Fractions

Isolated DIG fractions are treated with solvents extracting all lipid molecules (DIG lipids). The remaining protein fraction is referred to as DIG proteins. This methodology allow quantitative and qualitative analysis of both proteins and lipids associated with the isolated DIGs.

Proteins are analysed by proteomic including 2D gels, Western blot and masspectrometry.

### EXAMPLE 2

### Isolation and Characterisation of DIGs from H69 Small Cell Lung Cancer Cells

Gangliosides GM1 and Fuc-GM1 in DIGs and whole

| | | Protein | GM1 | Fuc-GM1 |
|---|---|---|---|---|
| | Number | total | (nmol/mg | (nmol/mg |
| | of cells* | (□g) | protein) | protein) |
| H69 DIG's | 50 × 10⁶ | 16 | 54.2 | 20.6 |
| H69 Total cell | 5 × 10⁶ | 600 | 0.7 | 0.3 |

Conclusion: Gangliosides are enriched 70-80 times in DIGs
*Starting material

Protein

The attached Figure shows a 2D gel showing DIGs associated proteins as compared to whole cell protein content. It is evident from the gel run that selected proteins are found in DIGs. These proteins comprise minor—almost non-detectable fractions in the whole cell protein fraction.

1. Method for treating cancer whereby a therapeutically effective immunogenic amount of at least one raft containing tumour protein(-s) and/or glycosphingolipid(-s), is administered to a mammal suffering from a cancer.

2. Method according to claim 1, wherein the at least one raft is administered as a source of cancer antigen in cancer immunotherapy.

3. Method for treating cancer whereby an antigen-presenting cell is first contacted with at least one raft containing tumour protein(-s) and/or glycosphingolipid(-s), and then administering said antigen-presenting cell or its stimulated immune cells is administered in a therapeutically effective amount to a mammal.

4. Therapeutical, immunogenic composition comprising at least one raft containing tumour protein(-s) and/or glycosphingolipid(-s) and/or an antigen contained therein to be used in cancer therapy.

5. Therapeutical, immunogenic composition comprising an antigen-presenting cell having been brought into a first contact with at least one raft containing tumour protein(-s) and/or glycosphingolipid(-s), and/or comprising said antigen-presenting stimulated immune cell in a therapeutically effective amount.

6. The use of lipid enriched domains isolated with any procedure being able to challenge the lipid-lipid intramembrane interaction from tumor cells or tumor tissues or of combinations of molecules found therein in the manufacture of a therapeutical immunogenic composition for tumor immunotherapy.

7. The use according to claim 8, wherein the composition is a vaccine.

8. The use according to claim 9, wherein the composition is an injectable vaccine.

9. The use according to claim 9, wherein the composition is an inhalable vaccine.

10. Method for identifying and isolating at least one raft, membrane domain, containing tumour protein(-s) and/or glycosphingolipid(-s), wherein cells or tissue specimens or cellular membranes therefrom are homogenized in solutions preserving DIGs as identified above, separating any DIGs from non-DIGs fractions by any methodology, where the lipid enriched characteristics are utilized, extracting isolated DIG fractions with solvents extracting all lipid molecules (DIG lipids) and leaving a remaining protein fraction referred to as DIG proteins.
